(19) United States
(12) Patent Application Publication
(10) Pub. No.: US 2022/0133227 A1
(43) Pub. Date: May 5, 2022

(54) METHOD FOR STRESS DETECTION UTILIZING ANALYSIS OF CARDIAC RHYTHMS AND MORPHOLOGIES

(71) Applicants: Jason Felix, Vashon, WA (US); Leslie Chertok, Vashon, WA (US); Vandana Verma, Mountain View, CA (US)

(72) Inventors: Jason Felix, Vashon, WA (US); Leslie Chertok, Vashon, WA (US); Vandana Verma, Mountain View, CA (US)

(73) Assignee: BioPause LLC, Vashon, WA (US)

(21) Appl. No.: 17/089,266

(22) Filed: Nov. 4, 2020

(51) Int. Cl. A61B 5/00; A61B 5/0408

(52) U.S. Cl. CPC A61B 5/4884; A61B 5/7246; A61B 5/683; A61B 5/0408; A61B 5/7264

(57) ABSTRACT

A method for detecting stress in organisms with a cardiac organ is provided. A cardiac waveform is input to an analysis system which decomposes the incoming signal to detect patterns within the decomposed segments. The patterns are comprised of one or more of the following: the overall waveform of a series of beats, a single beat or segments contained within a beat. The decomposed parts of the cardiac waveform are classified according to types of stress patterns both known in the art and dynamically learned through feedback. When the system detects that a sufficient threshold of stress has been exceeded, a notification can be generated and the details of the stress, such as the severity and type can be communicated to an external module, system, user or host. Patterns indicating a future or rapidly increasing stress level, signaled by evolving patterns in the cardiac waveform can be detected and an alert generated before a major or difficult to control stressful event is externalized by the organism.

### FIELD

This application relates in general to health and fitness monitoring and in general to the application of psychological and physiological stress detection.

### BACKGROUND

When the cardiac muscles contract and relax a biopotential voltage is generated which propagates through an organism's body. The fine details of the cardiac rhythm are as unique as a fingerprint; however, all organisms of the same species share some similarities in their cardiac rhythm.

The cardiac muscles contract and relax differently depending on the psychological and physiological condition of the organism which is widely triggered by the external stimulus which it is experiencing. Conditions such as age, disease, health, body mass, menstrual status and disease state create a baseline rhythm for the organism that corresponds to its physical and mental state. The baseline rhythm is typically modified when an organism experiences a stimulus which can be physical or mental, internal or external. The cardiac muscle group and electrophysiological control of the organism responds almost instantly to a stimulus—often far before the organism becomes conscious of the stimulus it has already started to react to. This reaction causes rhythm and morphology changes in the cardiac waveform which can indicate the magnitude and severity of stress, or even predict the onset of future stress.

Stress is a response to a stimulus that disrupts the physical, mental or emotional environment, resulting in physical and/or emotional strain. A stressful event has multiple consequences for an organism's physiology. One of the main ways a human body registers stress is through the “fight or flight” response. During a stressful environmental stimulus, a variety of physiological signals are registered by the human body. Adrenaline plays a critical role in this “flight or fight response” which prepares the body for strenuous activity under stressful conditions. Found in tiny amounts in the body and released in response to stress, adrenaline is essential for maintaining heart rate, diverting blood to specific tissue when responding to stressful events. Feelings and emotions such as fear (including freeze, faint, flee and fight) as well as anger can cause adrenaline to be released into the bloodstream. This rapidly leads to physiological changes such as an increase in heart rate, blood flow to muscles, changes in blood pressure, and sugar metabolism (Gu et al 2016). When stress is ongoing or chronically repeating, adrenaline is in constant production which, triggers a chronic stress response in the body leading to continuous cortisol production. Ongoing elevated levels of cortisol in the body have been shown to lead to disease and inflammation.

Most modern-day stressors are often not ‘physically threatening’. They are psychologically threatening, yet these psychological stressors create the same physiological adrenaline response that prepares an organism to respond physical threats. While an organism may not be consciously aware of its physiological response. Eventually, the organism may notice the psychological state created by constant stress and anxiety. Exposure to this chronic stress induces various physical, emotional and mental outcomes that can ultimately lead to sickness. Stress-related disorders in Humans are a global health problem that costs the US economy $190B each year. There is therefore a great need in the art for recognizing, decreasing frequency of stress responses and minimizing the negative impact of stress in individuals. Eustress is also a type of stress. As used herein, eustress means beneficial stress—either psychological, physical (e.g., exercise) or biochemical/radiological (hormesis). The term was coined by endocrinologist Hans Selye, consisting of the Greek prefix eu- meaning “good” and stress, literally meaning “good stress”. Typically, the stress referred to in this document is negative stress.

According to WebMD 75-90% of primary care doctor visits for humans are related to stress, yet only 3% of patients receive stress management help. Both large and small stresses may cause similar metabolic cascades in the human body. When an individual feels stressed they experience a reduced ability for making conscious choices (working memory has reduced capacity during the fear response). Under chronic stress, people can also become numb to stress and not recognize the impacts on their body and general wellbeing. The best technique to address stress and its impact is to notice as it arises and immediately employ countermeasures to alleviate it before it becomes unmanageable. Additionally, interrupting these patterns early and consistently can prevent them from becoming habitual reactive patterns. There is need therefore for a device that is able to accurately measure and detect increasing stress levels and to provide an alert or warning signal. The organism under stress can then learn ways to interrupt their individual stress reaction as well as to improve (lower) their stress baseline levels. One such countermeasure includes relaxation techniques to stimulate the parasympathetic nervous system and increase vagal tone, thereby reducing the amount of circulating adrenaline. It can also be improved through the healing/resolution of past traumatic events which contribute to the users stress baseline. There is need for noticing real time stress in the moment and providing feedback on the measure of stress that is elevated above a previous state or predefined level.

Most stress occurs during everyday activities; hence a need exists to measure stress during a normal routine as it is occurring in real time. Users need to be alerted to stress as it occurs so changes can be implemented to alter their physiological state immediately. When technology is used to assist in identifying the first signs of stress, a small intervention—a conscious breath, a subtle movement, a simple thought shift—can have a huge impact on the outcome. This also allows gradual learning of new patterns and allows implementation of new healthier responses, perhaps eventually with no further need for a prompt or alert system.

Recently there has been a great rise in the use of wearable devices, originating within the medical industry and now within the large consumer fitness wearable market. This market need has expanded to include many aspects of health and well-being. There has been a desire in the field to measure heart rhythms with wearable devices as individuals recognize the need for recording health information during regular activities. New technology has allowed smaller, longer lasting sensing devices that are practical for everyday use. The signals in extremities however, such as the wrist, where wearables are often placed are less accurate than on the placement on the torso, located in proximity to the heart. To be of greatest use, wearables need to be low power to allow for extended periods of use.

Heart Rate Variability or HRV is the physiological phenomenon of the variation in the time interval between successive heartbeats in mammals. In the field of stress management, it is used as a measure of stress in individuals. Many devices can measure HRV along with other physiological indicators to provide a picture of health and fitness of an individual. Most of these devices fitting this description use a wrist based photoplethysmographic optical sensor to measure HRV and other parameters. Such devices in the art include:

1. Fitbit tracker, measuring heart rate, sleep and exercise through wrist-based sensors.

2. The Whoop monitor measures Heart Rate Variability (HRV), Resting Heart Rate (RHR), and sleep. calibrated to a baseline. Recovery from exercise and training performance can be calculated each day using an algorithm and subscription-based service

3. HeartMath technology claims to measure Coherence, a HeartMath term for an optimal physiological state which aims to reduce stress, increase resilience, and promote emotional wellbeing. Coherence is measured through Heart Rate Variability (HRV).

4. Mightier is a bioresponsive video game platform that creates an “emotional playground” for children. The platform uses video type games which is intended to elevate the users heart rate. The user participation is then decreased until the user can use a defined relaxation and calming method to decrease resting heart rate. Once these parameters are within a defined range, the user is able to return to the game. Thus the user learns calming and stress reduction methods through game play.

5. The Apple Watch is a device that can measures heart rate, and HRV through an optical sensor, as well as a biopotential sensor located between the back of the watch and the crown.

6. The Garmin Fenix 6 is able to measure heart rate and also contains a pulse oximeter in a wrist-based device.

The cardiac waveform signals the progression of the electrical impulse through the heart and vasculature, moving from depolarization to repolarization through various ionic currents. These are translated to contraction and relaxation of the atria and ventricles to move blood around the heart through various movements of cardiac structures.

The cardiac waveform, therefore, provides large amount of information about the heart and the organism's physiological state, e.g. abnormalities in the QRS complex (segments of an isolated heart beat) are likely to indicate abnormalities that are related to ventricular physiology. Changes in the morphology or features within the cardiac waveform, changes in beat patterns or changes in rhythms are correlated to the occurrence of stress. A possible embodiment of the stress recognition and warning system could be a device that will detect these morphologies of the cardiac waveform that are causing a stressful state. Once the stressful situation is detected a user could be notified. In one embodiment, changes in U wave onset is monitored and correlated to the stress state of an organism. When the relative position of the U wave onset decreases relative the R-wave, that indicates the organism is experiencing stress. In another embodiment pattern matching and learning is used to determine the dimensions and regions of the cardiac waveform responsible for stress and determine the relationship of the waveform to stress and alert the user to implement a change to decrease their stress level. Many of these stress waveforms may be related to sympathetic activity of the heart and mediated by the vagus nerve.

One type of specific variation in the cardiac waveform are premature contractions. PVCs and PACs (premature ventricular or premature atrial or any early contractions) are common among the general population. Long runs of these premature contractions can sometimes be felt in the chest as heart palpitations or a flutter, but typically are not detectable to most organisms. During a premature ventricular contraction (PVC), the heartbeat is initiated by the Purkinje fibers rather than the SA node, which typically initiates the heartbeat. Given that PVCs and PACS occur before a regular heartbeat, there is a pause before the next regular heartbeat. Within the heart itself, PVCs increase the dispersion of the action potential configuration/duration (electrical waveform of the heart). At the cellular level are due to desynchrony of calcium currents within cardiac myocytes giving rise to an extra systole (contraction). Benign causes of pulse irregularity such as PACs and PVCs are common in the general population but can also be caused by disease or a congenital heart condition.

PVCS can be seen in people of all ages. Low occurrence of PVCs inversely proportional to age is considered benign, however frequent occurrences are considered to be strongly correlated with chronic stress, especially in young people. Young and healthy adults normally have few occurrences of PVCs in contrast to the older segments of the general population. Symptoms of premature cardiac contractions (atrial and ventricular) are associated with emotional stress, physical activity, dietary factors, and caffeine or other stimulant use. Premature ventricular contractions in children with structurally normal hearts are thought be generally benign especially originating during exercise, and usually resolve with no need for any medical intervention.

Certain types and rates of PVCs occur in the presence of cardiovascular disease; heart disease, including congenital heart disease, coronary artery disease, heart attack, heart failure and a weakened heart muscle (cardiomyopathy). These patients are likely to be monitored using Holter monitors. Non-threatening and non-disease state causes of pulse irregularity, such as PACs and PVCs, are common in the general population. These irregularities are often considered to be stress related.

A recent study has shown that even for brief periods, PVCs powerfully modulate cardiac vagal afferent neurotransmission and reduce parasympathetic efferent outflow to the heart. Using in vivo recordings, it was found that PVCs activated both mechano- and chemosensory neurons in the nodose ganglia (Salavatian et al. 2019). This suggests that reduction of the activity of parasympathetic nervous system is related to preparation of the body for stress. Changes in heart rate variability (HRV) associated with breathing (respiratory sinus arrhythmia) are known to be parasympathetically (vagally) mediated when the breathing rate is within the typical frequency range (9-24 breaths per minute; high-frequency HRV) (Kromenacker et al. 2018). Therefore, assessing PVCs and their contribution to a user's stress response can also be used as a measure of stress.

It has been shown that deep breathing at 6 breaths/min reduced the frequency of PVCs by at least 50% (Prakash et al., 2006). The beneficial effect of this deep breathing is attributed to vagal modulation of the sinoatrial and atrioventricular node, and can help reduce the level of stress experienced by an organism. Within a device, described in later sections, by using an algorithm that can measure PVC frequency, a notification will encourage slow breathing prompts upon detection of PVCs at a desired frequency or similar, thereby decreasing stress in a rapid manner.

Accurate reading of stress in animals by exclusive observation of the R wave and variability of time between R wave peaks (HRV) has limited diagnostic efficacy. The R wave timing can be affected by a variety of factors in which physiological intervention is not necessary. A fuller analysis of the cardiac waveform's composition, timing and morphologies are a much more efficacious indicator of stress.

Some devices use R wave analysis (typically HRV) to measure stress. This may be due in part that the PVCs are comprised of lower frequency, lower amplitude waveforms and are more difficult to detect (especially on the extremities). New advances in sensing technology such as the Cardiac Science mySense Heart allow accurate detection of PVCs and thus can factor their occurrence into an overall stress score. Combining traditional HRV sensing with PVC sensing and other cardiac measurements provides a more accurate indicator of psychological stress.

In summary, stress detection systems to date that rely on cardiac activity to measure stress typically observe the R to R wave interval and from that derive a measurement known as Heart Rate Variability (HRV). Some studies have suggested that heart rate variability can be related to stress, however the accuracy and specificity of this measurement as related to stress is questionable. A more accurate method to measure stress—especially stress caused by emotional stimuli is needed.

### SUMMARY

The invention is a method for measuring stress by detecting patterns in the cardiac waveform morphology. In the method, a waveform is acquired and then input to a decomposition and classification module. To facilitate recognition of stress patterns, the waveform is decomposed into rhythms, beats and segments.

Once separated, the decomposed waveform categories are classified and a statistical analysis is computed of their characteristics. This categorical and statistical analysis result is known as the decomposition data.

The decomposition data, containing the statistical analysis and classification is stored in a database and then later compared to known stress patterns. Optionally, the database may be seeded with known stress patterns that are common to a particular organism, group or other designator such as, but not limited to: age, nationality, morbidity condition and body type.

The database of stress may be augmented or “learn” as stressful patterns are classified by an external source such as a trainer, additional sensor (such as a scream or motion sensor) or under the organism's direct input that a stressful event has occurred).

If a comparison of recent incoming decomposed data to the database of stress patterns indicates a high enough correlation coefficient, the stress threshold value would be exceeded, indicating recognition of stress. Actions could be taken based on the threshold being exceeded, the magnitude of how much the value has been exceed, and for how long the value is exceeded.

One method of computing a stress score could be computed based on the correlation coefficient of current decomposition data with the database and the duration for which the correlation occurs. Many methods of determining a score are possible and could be weighted based upon the type of correlation, the amount of correlation, the duration of correlation and the sensitivity of the organism's psychological state to stress.

Some examples of actions that could occur when a stressful event is detected may be a log entry, notification of the user, notification of a friend or physician or triggering of another device or system. The severity or type of responding action could be based upon the severity of the stress, predefined thresholds set by the system or learned thresholds.

### BRIEF DESCRIPTION OF THE DRAWINGS

FIG. 1 is a diagram of a cardiac biopotential differential voltage plot of a single heart beat.

FIG. 2 is a diagram of a cardiac biopotential differential voltage plot of a series of heart beats (cardiac rhythm) which is classified as a normal sinus rhythm (NSR).

FIG. 3 is a diagram of a cardiac biopotential differential voltage plot of a series of heart beats (cardiac rhythm) that contains four normal beats and two ectopic beats.

FIG. 4 is perspective view showing a series of monitoring devices connected via electrodes to a human.

FIG. 5 is perspective view showing a series of monitoring devices connected via electrodes to a dog.

FIG. 6 is a flow diagram showing a method of detecting stress and providing a notification.

### DETAILED DESCRIPTION

A cardiac biopotential waveform is acquired 61 and presented to a decomposition and classification subsystem 62.

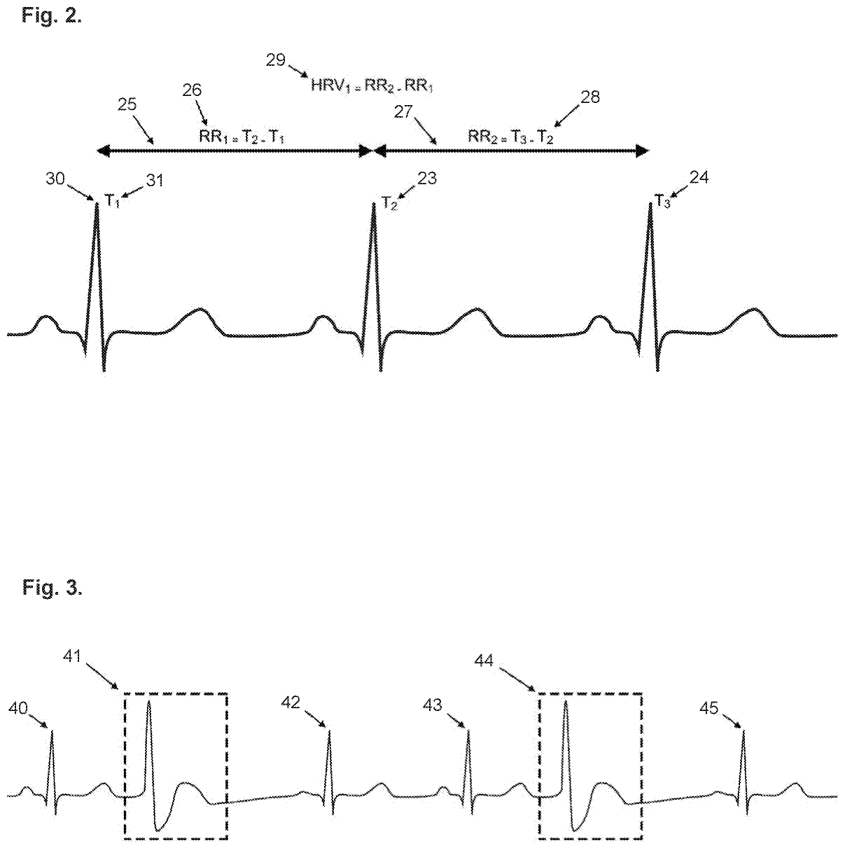

The decomposition and classification system 62 first detects segments of differing heart rhythms 63. Common examples may include normal sinus rhythm (NSR) FIG. 2, arterial fabulation (AFIB), ectopic rhythms FIG. 3, unknown rhythm or regions with no activity (pauses). The onset 30, offset 24, duration and other parameters (such as amplitude, frequency content, etc. . . . ) of each rhythm segment is calculated and stored 66.

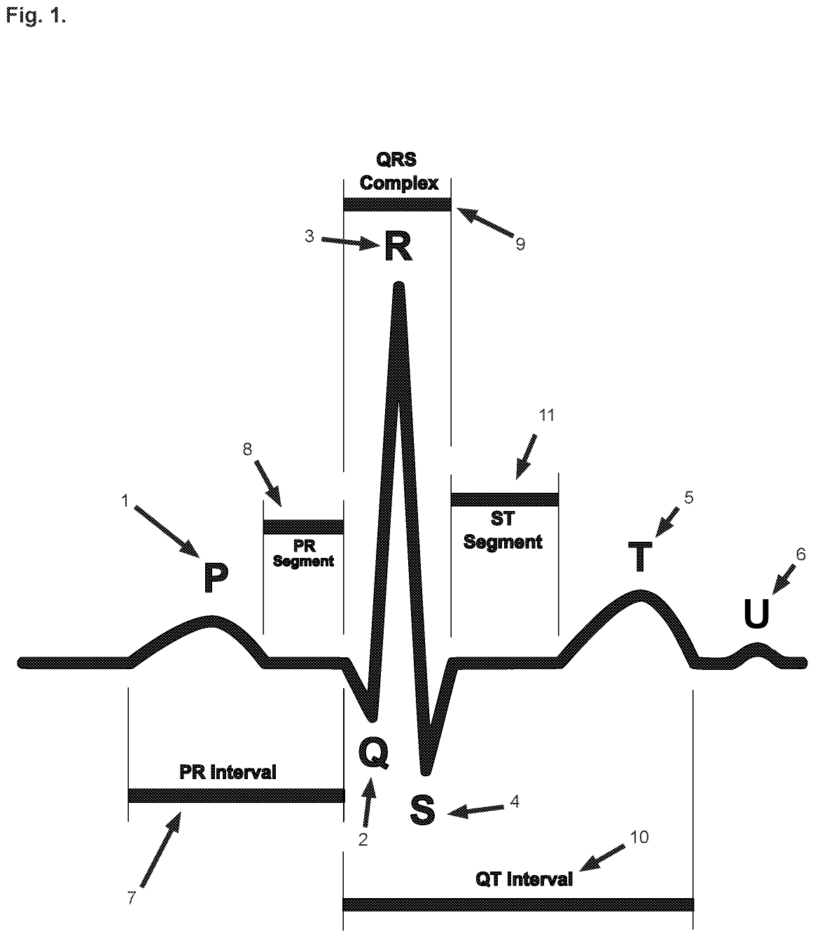

After the differing heart rhythms are separated and classified 63, the resulting segments are further split into beats FIG. 1 by the decomposition and classification system 64. The onset 40, offset 45, duration and other parameters (such as amplitude, frequency content, ectopic status etc . . . ) of each beat segment is calculated and stored 66.

After the differing beats are separated and classified 64, the resulting segments such as those shown in FIG. 1 are further split into smaller waves 1, 2, 3, 4, 5, 6 segments 8, 11, intervals 7, and complexes 9 that form the isolated beat by a segment classifier 65. The waves, segments, intervals and complexes are analyzed for parameters such as amplitude, frequency content, shape, presence, ectopic classification, order etc. . . . and sent to a storage system 66.

The storage system 66 receives the data from the waveform and decomposition system 62 and makes it available to the comparison and pattern recognition system 66.

The comparison and pattern recognition system 67 compares the decomposed and classified rhythms, beats and segment and determines if and how much they are similar to a database of rhythms, beats and segments of an organism under stress. The comparison and pattern recognition system 67 computes a correlation coefficient between the current activity captured by the waveform acquisition system 61 and known or user indicated stress patterns.

The correlation coefficient or “stress score” is checked against a threshold 68 to determine if a notification is needed. If the threshold exceeds a predetermined or dynamically computed value a stress notification is triggered 69.

The system may process additional waveforms as they are acquired 61. The system may optionally reset between acquisitions or optionally incorporate the organisms specific stress responses into the stress database 66 to better predict and recognize future stressful patterns as they occur again.

Viable physical implementations of the stress detection system are possible in a diverse array of configurations. FIG. 4 details several locations on the human body where cardiac biopotential measurements are possible. On the wrist 52 a device is shown similar to watch that performs the processing described above and alerts the user upon a trigger threshold being exceeded 65. On the left clavicle 51 a cardiac monitoring device such as the Zio Patch manufactured by iRhythm Technologies could be programmed to monitor for stress using the methods described in this patent and alert the using upon reaching a trigger threshold 69.

A device containing the stress monitoring method could be placed near the waist 50.

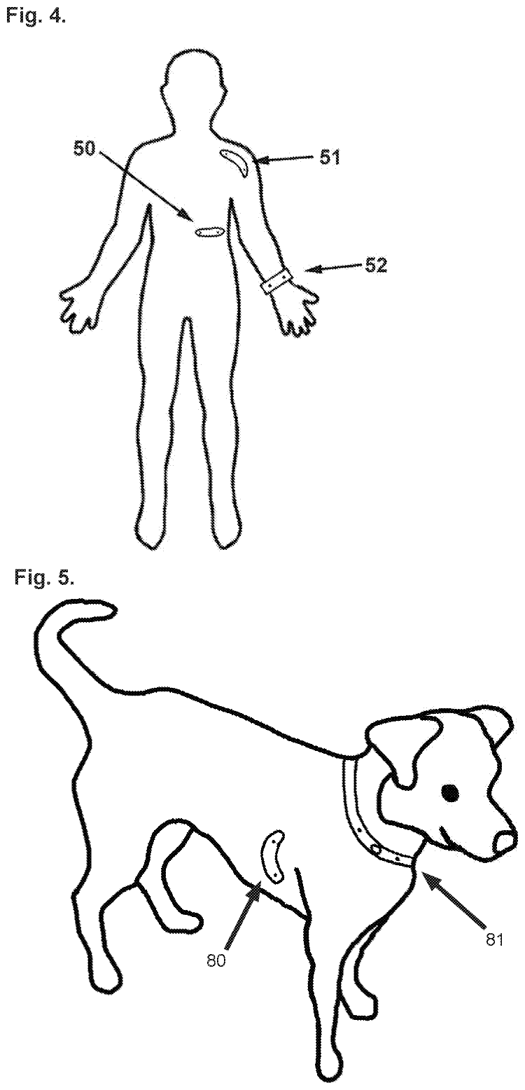

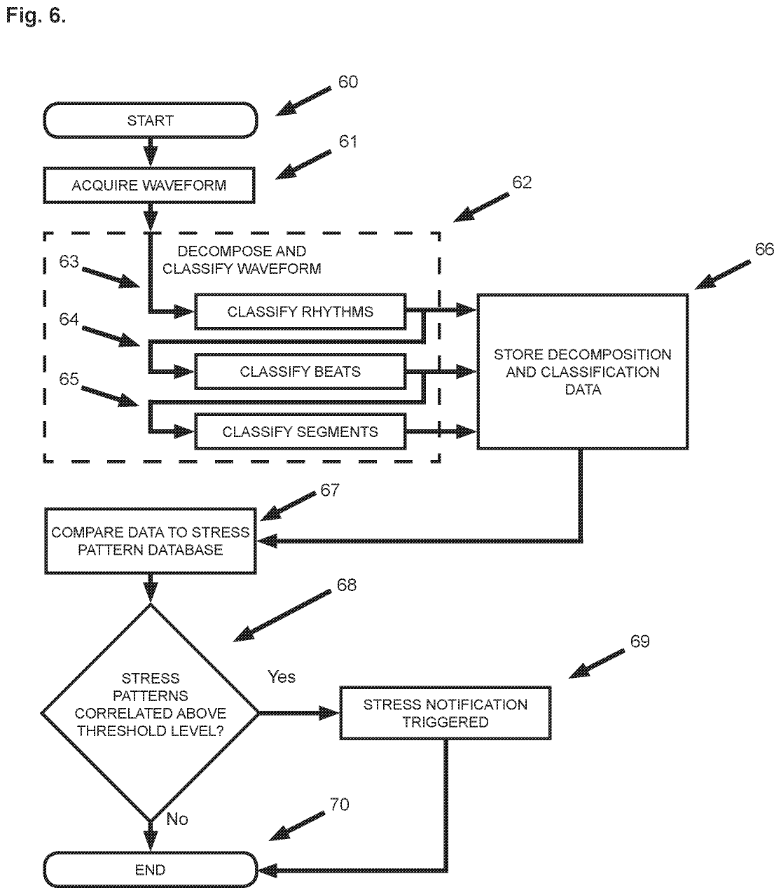

Stress monitoring is also valuable for non-human organisms such as pets. FIG. 5 shows a system embodiment that is implemented as a monitor worn on a collar 81 of a dog. Other embodiments are possible such as patches worn near the heart 80.

Still further embodiments are possible for a device that monitors stress using the methods described in this patent. Possible options for device placement include anywhere that the cardiac electro cardiogram is viable such as the back, chest and neck regions. Given a sensitive enough monitor, placement almost anywhere on an organism is possible.

In an additional embodiment of the system, the system is configured to analyze a specific type of ectopic beat known as a PVC and correlate that to an organism's stress level. To achieve this, stress patterns related to PVCs are preloaded into the stress database 67. A waveform such as the one in FIG. 3 is acquired 61 and sent to a waveform decomposition and classification system 62. The rhythm is classified 63 as sinus arrhythmia. The onset and offset of the sinus arrhythmia rhythm is calculated 62 and stored 66. The rhythm segment is next decomposed into beats and which are classified and analyzed 63. Information about the beats are extracted from the classification of the beats. The information may optionally include the type of beat, the time of the beat, the frequency of similarly classified beats, the amplitude of the beat, the timing relationship to other beats, and the timing relationship between ectopic and normal beats.

In this example, two PVCs 41, 44 are detected by the beat classification system. The amount of time between the PVCs 41, 44 is calculated as well as the number of PVCs 41, 44 relative to the number of normal beats 40, 42, 43, 45. The classification, quantity and timing information is stored 66 for later correlation with the stress pattern database 68.

In this particular embodiment, the beat segments 65 are not classified. Other embodiments may optionally choose to use this data for the purposes of stress recognition.

The Waveform Decomposition data (which is comprised of the classified rhythm information and classified beat information), that has been stored 66 is then compared to a stress pattern database 67. In this embodiment a predetermined threshold of a ratio of PVCs 41, 44 to normal beats 40, 42, 43, 45 has been pre-programmed into the threshold detection and triggering system 68. In this example the threshold for the ratio of normal beats to PVC has been exceeded and a stress notification is triggered 69.

1. A method of detecting stress employing correlation of cardiac waveform data to a stress pattern database.

2. The method of claim 1 wherein the method is configured to decompose and classify cardiac rhythms and correlate them against a stress pattern database for the detection of stress patterns.

3. The method of claim 1 wherein the method is configured to classify cardiac beats and correlate them against a stress pattern database for the detection of stress patterns.

4. The method of claim 1 wherein the method is configured to classify cardiac beat segments and correlate them against a stress pattern database for the detection of stress patterns.

5. The method of claim 1 wherein the method is configured to optionally classify cardiac rhythms, optionally classify cardiac beats or optionally classify cardiac beat segments and correlate them against a stress pattern database for the detection of stress patterns.

6. The method of claim 5 wherein the method is configured to detect patterns of ectopic beats and compare them against a stress pattern database for the detection of stress patterns.

7. The method of claim 5 wherein the method is configured to detect patterns of ectopic PVC beats and compare them against a stress pattern database for the detection of stress patterns.

8. The method of claim 5 wherein the method is configured to detect patterns of ectopic PAC beats and compare them against a stress pattern database for the detection of stress patterns.

9. The method of claim 5 wherein the method is configured to execute a special function when a stress threshold is exceeded.

10. The method of claim 5 wherein the method is configured to alert the user when a stress threshold is exceeded.

11. The method of claim 5 wherein the method is configured to alert a physician when a stress threshold is exceeded.

12. The method of claim 5 where in the method is configured to execute a physical action when a stress pattern is exceeded.

13. The method of claim 5 wherein an embodiment of the method is configured to reside on a flexible band containing surface monitoring electrodes.

14. The method of claim 5 wherein an embodiment of the method is configured to reside on a collar with surface monitoring electrodes.

15. The method of claim 5 wherein an embodiment of the method is configured to reside on a patch containing surface monitoring electrodes.

16. The method of claim 5 wherein an embodiment of the method is configured to be worn by a human.

17. The method of claim 5 wherein an embodiment of the method is configured to be worn by a non-human organism with a cardiac organ.

18. The method of claim 5 wherein an embodiment of the method is configured to be worn in conjunction with smart fabric.

19. The method of claim 5 wherein an embodiment of the method is adjusted with higher built in thresholds to be used in a predictively stressful state such as during counseling or surgery.
